(19) United States
(12) Patent Application Publication
(10) Pub. No.: US 2024/0100301 A1
(43) Pub. Date: Mar. 28, 2024

(54) TREATMENT CATHETER INCLUDING REMOVABLE HUB AND RELATED METHODS

(71) Applicant: Mohammad ALMUTAWA, Edmonton (CA)

(72) Inventor: Mohammad ALMUTAWA, Edmonton (CA)

(21) Appl. No.: 18/273,402

(22) PCT Filed: Jan. 20, 2022
(86) PCT No.: PCT/CA2022/050082

§ 371 Date: Jul. 20, 2023

Related U.S. Application Data
(60) Provisional application No. 63/139,866, filed on Jan. 21, 2021.

(51) Int. Cl. A61M 25/00; A61M 25/09

(52) U.S. Cl. CPC A61M 25/0097; A61M 25/09

(57) ABSTRACT

Treatment catheters including a removable hub, and methods for using such treatment catheters with guide extension catheters during a medical procedure are disclosed. A method can include positioning a treatment catheter over a guidewire and through a guide catheter such that a proximal end of the treatment catheter extends from a proximal end of the guide catheter. The method can include removing a removable hub from the proximal end of the treatment catheter and advancing a guide extension catheter having a tubular distal portion through the guide catheter and along an outer surface of the treatment catheter. Advancing the guide extension catheter can include sliding the tubular distal portion of the guide extension catheter over the hubless proximal end of the treatment catheter and reattaching the removable hub to the proximal end of the treatment catheter.

### CROSS REFERENCE TO RELATED APPLICATIONS

The present application claims priority to U.S. Ser. No. 63/139,866 filed Jan. 21, 2021, the contents of which are hereby incorporated by reference for all purposes.

### TECHNICAL FIELD

This patent document relates to medical devices. More particularly, but not by way of limitation, the patent document relates to catheter systems and methods.

### BACKGROUND

Coronary artery disease (“CAD”), also known as atherosclerosis or plaque buildup, can develop over time as blood vessels within the heart, specifically the coronary arteries, are narrowed or clogged. When blood vessels in the heart are narrowed, blood flow to regions of the heart muscle can be restricted and the muscle can be deprived of oxygen, leading to ischemia. Regions where a blood vessel in the heart is narrowed can be referred to as lesions or stenoses. In severe cases, a blood clot from a lesion can completely block the flow of blood through a vessel, leading to a myocardial infarction (“heart attack”). In these cases, the patient's life may be at risk, so it is imperative that the blood vessel be re-opened quickly (i.e., within hours of the blockage). For the purposes of this application, the term “vessel” is to be understood to include but not be limited to blood vessels, arteries, coronary arteries, and the like.

### OVERVIEW

Treatment of severe lesions and complete blockages may involve re-opening of the vessel at the blocked region or bypassing the blockage to re-establish normal blood flow. The most common treatment for CAD is dilation of the blood vessel. For the purposes of this application, the term “treatment catheter” is to be understood to include but not be limited to balloon catheters, stent catheters, microcatheters, and other treatment catheters. A guide catheter can be advanced into an ostium of an artery having a lesion to be treated. A guidewire can then be advanced through the guide catheter and into the artery or vessel to provide a stable access route for treatment catheters. A treatment catheter can be advanced over the guidewire and within the guide catheter to position a balloon or stent, for example, across the lesion. However, if the lesion is particularly tight it can be difficult to advance the treatment catheter across the lesion. Tighter lesions can require an increased force on the treatment catheter to cross the lesion, but an increased reactive force can cause the guide catheter to “back out” of the vessel ostium. If the reactive force is high enough, the guide catheter can continue to back out, causing the treatment catheter to buckle and preventing treatment of the lesion.

One approach that has been tried in scenarios where the guide catheter is prone to backing out is to “deep seat” the guide catheter by advancing its distal tip more deeply into the vessel's ostium. While this maneuver can increase the anchoring force of the guide catheter, it risks causing damage to a proximal portion of the vessel. The high relative stiffness of the guide catheter (compared to the stiffness of the distal portion of a guidewire or treatment catheter, for example) can scrape or dissect the vessel, which are both very serious complications. As a result, this technique is rarely performed today.

More recent developments have produced guide extension catheters. Guide extension catheters are tubular at a distal end and can be configured to move along a guidewire during advancement and/or placement of the guide extension catheter and to allow for subsequent catheter devices, such as treatment catheters, to be advanced through the guide extension catheter. A distal shaft section of the guide extension catheter is designed to be flexible such that it can safely be advanced deep into an artery or vessel. Standard guide catheters are relatively rigid in comparison to guide extension catheters, and therefore the risk of complication is higher when deep seating a conventional guide catheter as compared to a guide extension catheter. In use, a guide extension catheter is advanced through a guide catheter along a guidewire and into a coronary artery.

Guide extension catheters are often used on an “as needed” basis, after attempts have been made to perform a procedure with other interventional devices (for example, a treatment catheter) and in the absence of a guide extension catheter. When used after attempts have been made with other interventional devices, the previously used interventional device(s) is removed from the guide catheter before the guide extension catheter can be used. When the treatment catheter, for example, is removed, the guidewire is “alone” in the guide catheter, risking loss of guidewire positioning. Further, the treatment catheter needs to be repositioned after the guide extension catheter is in place, increasing the number of steps and duration of the procedure.

The present inventor recognizes that rather than having to entirely remove a treatment catheter from a guide catheter in order to use a guide extension catheter in cases when the guide extension catheter is used “as needed”, it would be desirable for an operator to be able to deliver a guide extension catheter without removing the treatment catheter from the guide catheter.

The present treatment catheters can include a catheter body and a removable hub secured at a proximal end of the catheter body. The treatment catheter can be advanced through a guide catheter to treat a lesion in a coronary artery. The guide catheter can be configured to receive the treatment catheter at a proximal end of the guide catheter. The treatment catheter can move axially along a guidewire within a lumen of the guide catheter, and the proximal end of the treatment catheter can extend from a proximal end of the guide catheter. When a guide extension catheter is to be utilized as part of the procedure, the removable hub of the treatment catheter can be removed to allow insertion of the guide extension catheter within the lumen of the guide catheter. The guide extension catheter can be configured to slide over the hubless proximal end of the treatment catheter and move distally along the treatment catheter through the guide catheter to exit the guide catheter at a distal end of the guide catheter without the need to withdrawal the treatment catheter from the guide catheter. The treatment catheter can be further advanced to exit the distal end of the guide extension catheter into the coronary artery.

A method can include positioning the distal end of a guide catheter within the ostium of a coronary artery, for example. A guidewire can be advanced through the guide catheter to a region to be treated. A treatment catheter can be placed over an exposed portion of the guidewire at the guidewire's proximal end, inserted into the guide catheter, and advanced distally along the guidewire, where a proximal portion of the treatment catheter extends from a proximal end of the guide catheter. A removable hub at a proximal end of the treatment catheter can be removed, and a guide extension catheter can be advanced over the hubless proximal end of the treatment catheter. The guide extension catheter can then advance distally through the guide catheter until a portion of the guide extension catheter extends from a distal end of the guide catheter. Once the guide extension catheter is positioned, the removable hub can be resecured, and the treatment catheter can be advanced deeper into the coronary artery with the support of the guide extension catheter.

According to an aspect of the invention is a method comprising:
positioning a treatment catheter over a guidewire and through a guide catheter such that a proximal end of the treatment catheter extends from a proximal end of the guide catheter;
removing a removable hub from the proximal end of the treatment catheter;
advancing a guide extension catheter having a tubular distal portion through the guide catheter and along an outer surface of the treatment catheter, including sliding the tubular distal portion of the guide extension catheter over a hubless proximal end of the treatment catheter; and
reattaching the removable hub to the proximal end of the treatment catheter.

According to a further non-limiting aspect is a treatment catheter, comprising:
a catheter body; and
a removable hub secured to a proximal end of the catheter body,
wherein the removable hub is removable from the catheter body to allow for advancement of a guide extension catheter over the proximal end of the catheter body.

According to further non-limiting aspects the treatment catheter as described herein is for treatment of a vessel lesion and/or vessel blockage.

According to further non-limiting aspects the treatment catheter as described herein is for treatment of a diseased vessel, blocked vessel, or hollow structure. In non-limiting aspects the hollow structure is biliary tract, ureter, and the like.

According to further non-limiting aspects the treatment catheter as described herein is for treatment of coronary artery disease.

According to a further non-limiting aspect, is a treatment catheter comprising a catheter body and a removable hub secured to a proximal end of the catheter body,
wherein the removable hub is removable from the catheter body to allow for advancement of a guide extension catheter over the proximal end of the catheter body,
wherein said treatment catheter is for positioning over a guidewire and through a guide catheter such that a proximal end of the treatment catheter extends from a proximal end of the guide catheter;
wherein a removable hub is removable from the proximal end of the treatment catheter;
wherein a guide extension catheter having a tubular distal portion is for advancing through the guide catheter and along an outer surface of the treatment catheter, including sliding the tubular distal portion of the guide extension catheter over a hubless proximal end of the treatment catheter; and
wherein the removable hub is reattachable to the proximal end of the treatment catheter.

These and other examples and features of the present systems, devices, and methods will be set forth, at least in part, in the following Detailed Description. This Overview is intended to provide non-limiting examples of the present subject matter—it is not intended to provide an exclusive or exhaustive explanation. The Detailed Description below is included to provide further information about the present systems, devices, and methods.

### BRIEF DESCRIPTION OF THE DRAWINGS

In the drawings, like numerals can be used to describe similar features and components throughout the several views. The drawings illustrate generally, by way of example but not by way of limitation, various system, device, and method embodiments discussed in this patent document.

FIG. 1. illustrates a plan view of a guide catheter advanced through an aorta to an ostium of a coronary artery, as constructed in accordance with at least one embodiment.

FIGS. 2A-B. illustrate, respectively, a guide catheter having its distal end positioned in the ostium of a coronary artery, and a guide catheter and guide extension catheter positioned in the coronary artery.

FIG. 3. illustrates an elevational view of an example rapid exchange guide extension catheter for use with a guide catheter, a guidewire, and a treatment catheter, as constructed in accordance with at least one embodiment.

FIG. 4. illustrates a plan view of an example treatment catheter having a removable catheter hub and a hemostasis valve, as constructed in accordance with at least one embodiment.

FIGS. 5A-B. illustrate an example treatment catheter having a removable catheter hub with a movable lever, as constructed in accordance with at least one embodiment.

FIGS. 6A-B. illustrate an example treatment catheter having a removable catheter hub with a threaded connection between two portions of the removable catheter hub, as constructed in accordance with at least one embodiment.

FIGS. 7A-D. illustrate example removable catheter hubs, as constructed in accordance with at least one embodiment.

FIG. 8. illustrates a plan view of the guide extension catheter of FIG. 3 used with a guide catheter and a treatment catheter having a removable hub, as constructed in accordance with at least one embodiment.

FIG. 9. is a flowchart of example operations for delivering a guide extension catheter over a prepositioned treatment catheter, in accordance with embodiments of the present invention.

FIGS. 10A-C. illustrate plan views of catheters positioned in a coronary artery corresponding to the operations of the flowchart of FIG. 9, as constructed in accordance with at least one embodiment.

The drawing figures are not necessarily to scale. Certain features and components may be shown exaggerated in scale or in schematic form, and some details may not be shown in the interest of clarity and conciseness.

### DETAILED DESCRIPTION

Oftentimes, after a treatment catheter or other interventional coronary device is inserted into a patient via a guide catheter, it can be necessary to withdraw the treatment catheter or device to use a guide extension catheter. For example, the treatment catheter can sometimes be difficult to advance across a diseased site (e.g., a stenosis) to be treated if the diseased site is especially narrow or tight. If the operator applies a force to advance the treatment catheter, the guide catheter may become dislodged from a vessel ostium. When this occurs, a guide extension catheter may be used to advance the treatment catheter in a coronary artery so that the diseased site can be crossed and properly treated. In operation, the treatment catheter is removed from the guide catheter and the guide extension catheter is inserted. Once the guide extension catheter is positioned, the treatment catheter is reinserted and advanced into the coronary artery and across the diseased site with the support of the guide extension catheter. The presence of the guide extension catheter provides additional crossing support to reduce the likelihood that the guide catheter will be dislodged or “back out” from the ostium of the coronary artery while directing the treatment catheter across the tight stenosis.

The present systems, devices, and methods allow for insertion of a guide extension catheter after a treatment catheter has already been inserted without the need for removal of the treatment catheter. Inserting the guide extension catheter without removing the treatment catheter reduces the duration of the procedure, as the treatment catheter, and potentially an interventional device (e.g., guidewire) internal to the treatment catheter, does not need to be removed and repositioned.

It is believed that the present systems, devices, and methods will find utility by interventional cardiologists performing coronary interventions with treatment catheters, particularly when a guide extension catheter is used “as needed.” Although the remainder of this patent document generally discusses and illustrates use of the present systems, devices, and methods with reference to treating coronary vessels, it should be understood that the devices and methods can also be used for treating other diseased or blocked vessels or other hollow structures (e.g., biliary tract, ureter, etc.) throughout a patient's body where guidewires are employed.

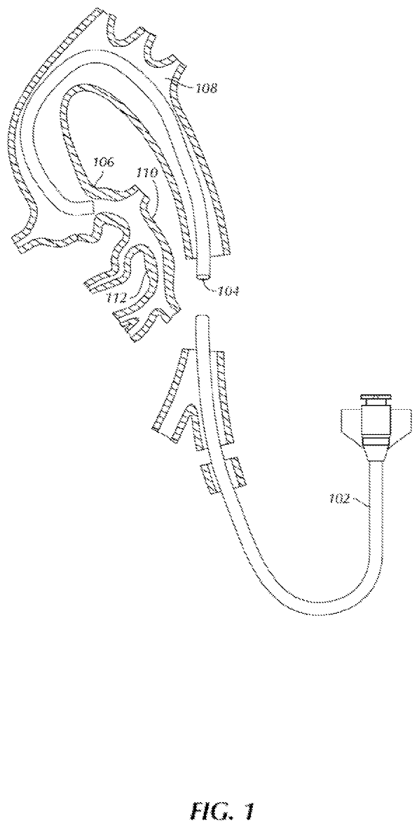

Minimally invasive cardiac interventions, such as coronary angioplasty procedures, are utilized throughout the world and typically include the use of a guide catheter. FIG. 1 illustrates a plan view of a guide catheter advanced through an aorta to an ostium of a coronary artery, as constructed in accordance with at least one embodiment. A guide catheter 102 is an elongate tubular member defining a guide catheter lumen 104 throughout its length. The guide catheter 102 can be formed of polyurethane, for example, and can be shaped to facilitate its passage to a coronary ostium 106 or other region of interest within a patient's body. In the example of FIG. 1, the guide catheter 102 can be inserted at a femoral artery and advanced through an aorta 108 to a position adjacent to the ostium 106 of a coronary artery 110. The diameter and rigidity of the guide catheter 102 oftentimes does not permit it to be advanced beyond the ostium 106 into the coronary artery 110 requiring treatment, and thus, a treatment catheter must be advanced independently of the guide catheter 102 to reach a diseased site 112.

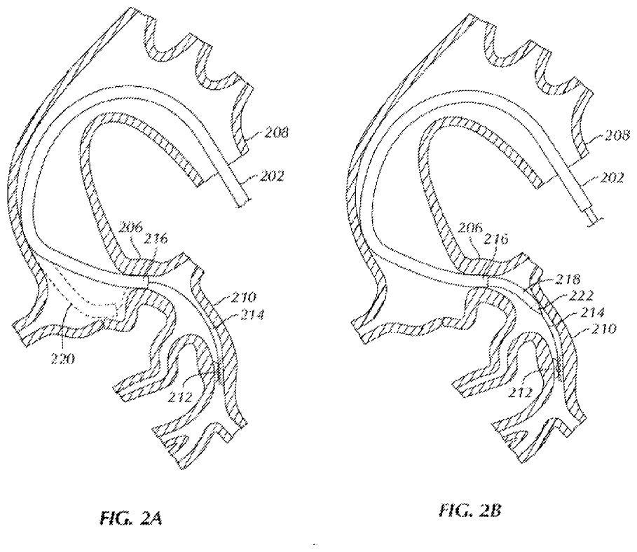

Maintaining the position of the guide catheter's 102 distal end at the ostium 106 can be desirable to facilitate the treatment catheter successfully reaching the diseased site 112. When resistance is encountered as attempts are made to deliver the treatment catheter, or an interventional device (e.g., guidewire), through the guide catheter 102, the guide catheter 102 can “back-out” or withdraw from the ostium 106, as discussed below with reference to FIGS. 2A-2B. A heart's intrinsic beat can also cause the guide catheter's 102 distal end to lose its positioning or otherwise be shifted so that it no longer is positioned to guide the treatment catheter to the diseased site 112. Because of this shift away from the ostium 106, access to the coronary artery 110 and the diseased site 112 can require repeated repositioning of the guide catheter 102 or selection of a replacement guide catheter having a different-shaped distal curvature in order to bring its distal end back into engagement with the ostium 106.

Numerous variables can impact how easy or difficult it is to treat a particular patient's lesion. Many of these variables relate to anatomical variation of a particular patient's aortic or coronary vascular anatomy. As such, guide extension catheters may be used later in a procedure on an “as needed” basis, rather than planning to use a guide extension catheter prior to the procedure, after a physician unsuccessfully attempts to deliver a treatment catheter or other interventional device.

FIGS. 2A-2B illustrate, respectively, a guide catheter having its distal end positioned in the ostium of a coronary artery, and a guide catheter and guide extension catheter positioned in the coronary artery. FIG. 2A depicts a guide catheter 202 passing through an aorta 208 to an ostium 206 of a coronary artery 210. FIG. 2A also depicts a guidewire 214 passing through the guide catheter 202 and into the coronary artery 210. Located in the coronary artery 210 is a lesion 212 to be treated. In a typical procedure, the guidewire 214 is placed through the aorta 208 and into the ostium 206 of the coronary artery 210. The guide catheter 202 is passed over the guidewire 214 until a distal end 216 of the guide catheter 202 is seated in the ostium 206 of the coronary artery 210. Force is then applied to the guidewire 214 to advance the guidewire 214 to the lesion 212. Once the guidewire 214 is at the lesion 212, a treatment catheter, sometimes including an interventional device (for example, a stent or a balloon) on its distal portion, can be passed along the guidewire 214 to the lesion 212.

When the treatment catheter is advanced to the lesion 212, a reactive force may be generated. If the lesion 212 is particularly tight (for example, coronary artery 210 has a small diameter residual lumen or is heavily calcified), it may be difficult to advance the interventional device across the lesion 212. The application of force to the treatment catheter can cause the guide catheter 202 to dislodge from the ostium 206 of the coronary artery 210. If the reactive force is high enough, it can cause the guide catheter 202 to completely “back-out” of the ostium 206. Dashed lines 220 depict a possible position of the guide catheter 202 that has backed out of the ostium 206.

FIG. 2B depicts the guide catheter 202 as used with the guidewire 214 and a guide extension catheter 218. The guide extension catheter 218 is passed through the guide catheter 202 and over the guidewire 214 into the coronary artery 210 after the guide catheter 202 has been placed in the ostium 206 of coronary artery 210. In some embodiments, the guide extension catheter 218 can include a tapered inner member 222 to allow the guide extension catheter 218 to enter the coronary artery 210 with increased ease. The guide extension catheter 218 can be positioned such that its distal end extends beyond the distal end 216 of the guide catheter 202. Once the guide extension catheter 218 is in place, the guide extension catheter 218 can accept a treatment catheter (not shown), such as a stent or balloon catheter, at a proximal end.

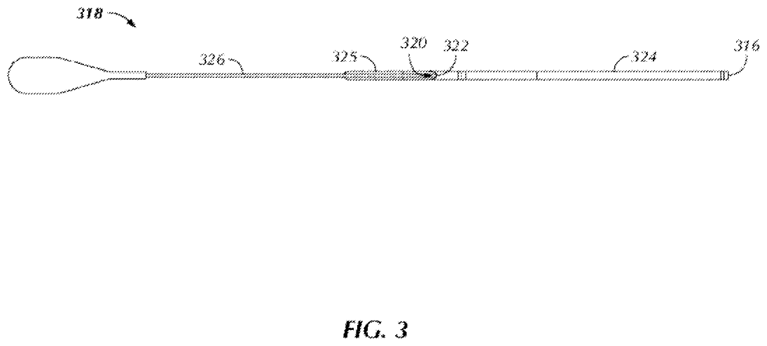

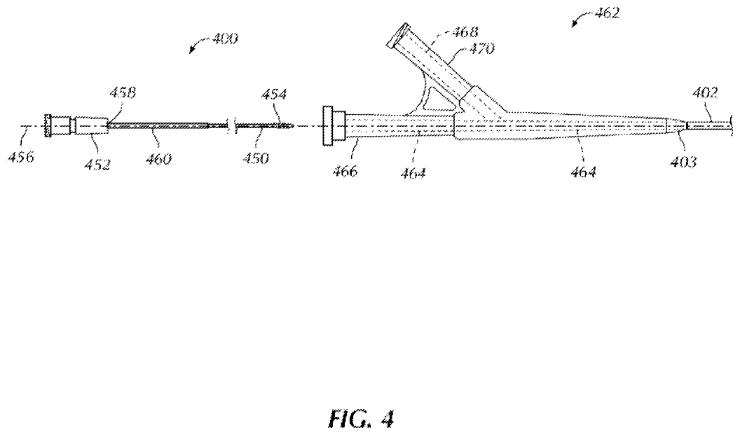

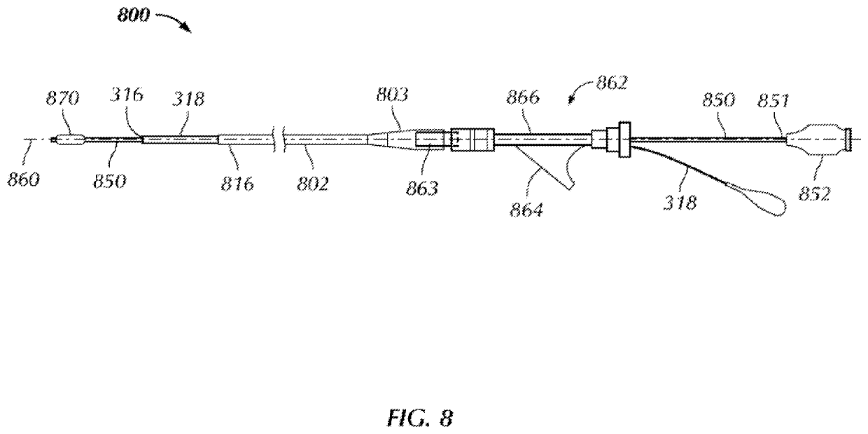

FIG. 3 illustrates an elevational view of an example rapid exchange guide extension catheter 318 for use with a guide catheter, a guidewire, and a treatment catheter, as constructed in accordance with at least one embodiment. A rapid exchange guide extension catheter 318 can be inserted into a guide catheter, such as guide catheter 802 as depicted and discussed below with reference to FIG. 8. Referring again to FIG. 3, the guide extension catheter 318 can have a distal shaft portion 324 and a proximal push member 326. The distal shaft portion 324 can include a reinforcement braid or coil (not pictured) to prevent kinks in the distal shaft portion 324 when the guide extension catheter 318 is advanced through an artery. The distal shaft portion 324 of the guide extension catheter 318 can be less rigid than a guide catheter in which it is inserted such that the guide extension catheter 318 is more flexible than the guide catheter.

The distal shaft portion 324 can be tubular and can have an inner lumen 320 configured to ride over a guidewire. For example, with reference to FIG. 2B, the guide extension catheter 218 can be advanced through the guide catheter 202 along the guidewire 214. Referring again to FIG. 3, the distal shaft portion 324 can include a lubricious inner liner along the inner lumen 320 to allow treatment catheters to advance through the guide extension catheter 318 to exit a distal end 316 of the guide extension catheter 318 with less force. The lubricious inner liner can also allow the guide extension catheter 318 to be advanced along a guidewire and/or an prepositioned treatment catheter (not pictured in FIG. 3). The distal shaft portion 324 can also include a lubricious external coating (e.g., a hydrophilic coating) to decrease the force required to advance the guide extension catheter 318 through the guide catheter.

Optionally, the guide extension catheter 318 can include an inner member having a tapered distal end to aid in delivering the guide extension catheter 318 to a diseased site in a narrowed artery. For example, with reference to FIG. 2B, the guide extension catheter 218 includes the tapered inner member 222. Referring again to FIG. 3, the distal end 316 can be soft as to not cause damage to the artery through which it is advanced. An outer diameter of the distal shaft portion 324 is less than an inner diameter of the guide catheter into which the guide extension catheter 318 is inserted. An inner diameter of the distal shaft portion 324 is larger than an outer diameter of a treatment catheter or other interventional device (not pictured) that is positioned within a guide catheter and that is advanced through, wholly or at least partially, the guide extension catheter 318 into a vessel. In some embodiments, the inner diameter of the distal shaft portion 324 can be approximately one French size smaller than the inner diameter of the guide catheter to maximize the number of treatment catheters and other interventional devices that can fit within and be advanced through the guide extension catheter 318.

The proximal push member 326 can be formed of a rod having a stiffness greater than a stiffness of the distal shaft portion 324. The greater stiffness of the proximal push member 326 as compared to the distal shaft portion 324 can allow for advancement of the distal shaft portion 324 through the guide catheter and into a vessel. The proximal push member 326 can be made from metal or any other substantially rigid material. For example, the proximal push member 326 can be a stainless-steel rod or ribbon. An outer dimension of the proximal push member 326 can be less than the outer diameter of the distal shaft portion 324 such that the proximal push member 326 does not interfere with devices that may be advanced alongside the guide extension catheter 318 within the guide catheter.

The guide extension catheter 318 can have a transition portion 325 between the proximal push member 326 and the distal shaft portion 324. The transition portion 325 can include a side opening 322 that transitions into the inner lumen 320 of the distal shaft portion 324. The transition portion 325 can include polymers of differing rigidity to allow for a secure and flexible coupling between the more flexible distal shaft portion 324 and the stiffer proximal push member 326. The varying flexibility of the transition portion 325 can decrease the potential for kinking and deformation when the guide extension catheter 318 is advanced through a guide catheter and subsequently into an artery.

A guide extension catheter having a combination of a relatively more rigid proximal push member with a relatively more flexible distal shaft portion can increase the “pushability” responsiveness of the guide extension catheter without sacrificing trackability of the distal shaft. While FIG. 3 depicts the guide extension catheter as a rapid exchange guide extension catheter, the guide extension catheter can alternatively be an over-the-wire catheter. The combination of a guide catheter with a guide extension catheter can provide improved distal anchoring of the guide catheter and the guide extension catheter within a coronary artery. The presence of the guide extension catheter within the guide catheter can also provide stiffer crossing support for a guidewire and/or a treatment catheter than would be provided by the guide catheter alone. The combination of the improved distal anchoring and stiffening of the guide catheter/guide extension catheter provides additional support to resist dislodging of the guide catheter from the ostium when a force is applied to a guidewire or treatment catheter to cross a lesion. In addition, the improved crossing support assists in the positioning of a stent or balloon positioned on the treatment catheter.

While the use of a guide extension catheter provides many benefits, various procedures as currently practiced include removal of the treatment catheter prior to insertion of the guide extension catheter. The present teachings allow for insertion of a guide extension catheter, via a removable hub of the treatment catheter, after the treatment catheter has been inserted and without removal of the treatment catheter. An advantage of using a guide extension catheter with a treatment catheter having a removable hub includes reducing the duration of the procedure, as the treatment catheter, and potentially an interventional device (e.g., a guidewire) internal to the treatment catheter, does not need to be removed and repositioned in order to use the guide extension catheter.

FIG. 4 illustrates a plan view of an example treatment catheter having a removable catheter hub and a hemostasis valve into which the treatment catheter is advanced, as constructed in accordance with at least one embodiment. A treatment catheter 400 includes a catheter body 450 and a removable hub 452. The catheter body 450 is tubular and includes an inner lumen. Optionally, the treatment catheter 400 can include a strain relief 460 located at a juncture of the catheter body 450 and the removable hub 452. The treatment catheter 400 can be a torqueable microcatheter and can include a screw portion 454 with external helical threads that, when advanced through an artery with rotation, engage a blockage in the artery. As depicted, the screw portion 454 is located distally followed by the catheter body 450 and the removable hub 452 located proximally. The treatment catheter 400 can be inserted into a guide catheter 402 via a hemostasis valve 462 connected to a proximal end of the guide catheter 402. For example, the proximal end of the guide catheter 402 can be connected to the hemostasis valve 462 with a rotating Luer lock 403. The hemostasis valve 462 includes a main lumen 464 defined by a linear branch 466 and a side lumen 468 defined by a side branch 470. When the treatment catheter 400 is inserted into the guide catheter 402, a proximal end 458 of the catheter body 450 can extend proximal of the proximal end of the guide catheter 402 and the hemostasis valve 462.

The removable hub 452 can be removed from the catheter body 450 to advance a guide extension catheter over the catheter body 450 and within the guide catheter 402. The removable hub 452 can be secured to the catheter body 450 via a threaded connection. For example, the proximal end 458 of the catheter body 450 can be externally threaded to mate with internal threads in the removable hub 452, and the removable hub 452 can be removed from the catheter body 450 by rotating the removable hub 452 relative to the catheter body 450.

The removable hub 452 can be attached to the catheter body 450 such that transverse and axial movement between the catheter body 450 and the removable hub 452 is inhibited. In some embodiments, the connection between the removable hub 452 and the catheter body 450 is a fluid tight connection. For example, the catheter body 450 can include a seal within an inner lumen at the proximal end 458 configured to receive a stem of the removable hub 452 and create a fluid tight seal around the stem of the removable hub 452. The seal can maintain an inflation fluid pressure within a balloon of a treatment catheter. For example, a balloon positioned at a distal end of the catheter body 450 can be advanced within a lesion and inflated. When the removable hub 452 is removed from the catheter body 450, the seal is actuated and maintains the inflation fluid pressure within the inner lumen of the catheter body 450 and the balloon. In other embodiments, the removable hub 452 can be secured to the catheter body 450 using any fastening method that permits a guide extension catheter (not shown) having an inner diameter larger than an outer diameter of the catheter body 450 to slide over the catheter body 450 when the removable hub 452 is removed, such that the guide extension catheter's distal shaft and the catheter body 450 are substantially coaxial with respect to a longitudinal axis 456. Example treatment catheters and removable hubs are now described.

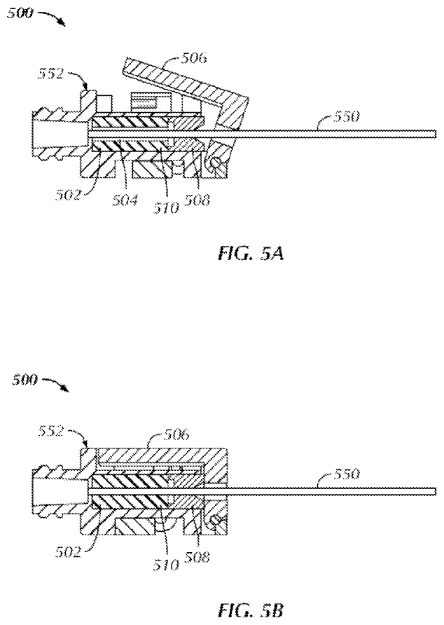

FIGS. 5A-B illustrate an example treatment catheter having a removable catheter hub with a movable lever, as constructed in accordance with at least one embodiment. An example treatment catheter 500 includes a removable hub 552 and a catheter body 550. The removable hub 552 includes a compressible plug 502 having a bore 504 positioned in the removable hub 552 and a lever 506 pivotable parallel to a longitudinal axis defined by the catheter body 550. To secure the removable hub 552 to the catheter body 550, the lever 506 is moved to an open position, as depicted by FIG. 5A, and a proximal end of the catheter body 550 is inserted into the bore 504 of the compressible plug 502. The lever 506 is then moved to a closed position, as depicted by FIG. 5B, where a plunger 508 applies an axial force to a distal end 510 of the compressible plug 502, radially deforming the compressible plug 502 inward and around the proximal end of the catheter body 550 to secure the catheter body within the removable hub 552. To remove the removable hub 552, the lever 506 is moved to the open position, as depicted in FIG. 5A, where the compressible plug 502 is decompressed and the removable hub 552 can be removed from the proximal end of the catheter body 550 by moving the removable hub 552 proximally relative to the catheter body 550.

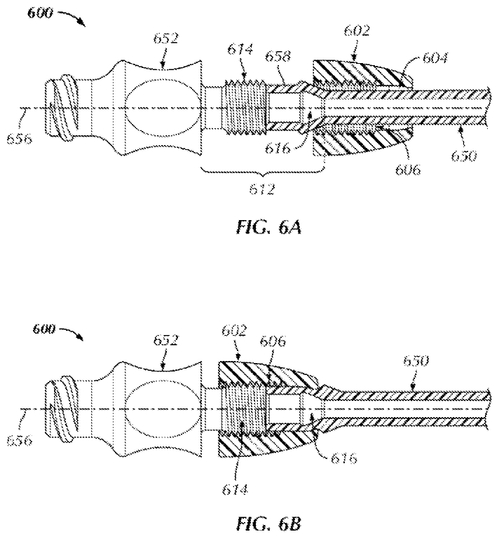

FIGS. 6A-B illustrate an example treatment catheter having a removable catheter hub with a threaded connection between two portions of the removable catheter hub, as constructed in accordance with at least one embodiment. An example treatment catheter includes a removable hub 600, and a catheter body 650. The removable hub 600 is made of two portions, a securing collar 602 and a coupling hub 652. The securing collar 602 includes a channel 604 with internal threads 606. The coupling hub 652 includes a connection stem 612 having external threads 614 and a barb 616. To secure the catheter body 650 within the removable hub 600, first a proximal end 658 of the catheter body 650 is fed through the channel 604 of the securing collar 602. The securing collar 602 is advanced along the catheter body 650 such that it is positioned distal of the proximal end 658 of the catheter body 650. Next, the barb 616 is inserted into the proximal end 658 of the catheter body 650, as depicted by FIG. 6A, such that the connection stem 612 is substantially coaxial with respect to a longitudinal axis 656. The securing collar 602 is then axially moved towards the coupling hub 652 and over the barb 616 until the internal threads 606 within the channel 604 engage the external threads 614 of the connection stem 612, as depicted in FIG. 6B. The securing collar 602 is then rotated relative to the catheter body 650 until the internal threads 606 of the securing collar 602 are fully engaged with the external threads 614 the coupling hub 652. To remove the removable hub 600, the securing collar 602 can be rotated relative to the catheter body 650 until the external threads 614 of the coupling hub 652 are disengaged from the internal threads 606 of the securing collar 602. The barb 616 can be removed from the proximal end of the catheter body 650 by applying an axial force to the coupling hub 652 such that the coupling hub 652 is moved proximally and the barb 616 is removed from the catheter body 650. The securing collar 602 can then be removed by proximally moving the securing collar 602 along the catheter body 650 such that the securing collar 602 is removed from the catheter body 650 as it moves over the proximal end 658 of the catheter body 650.

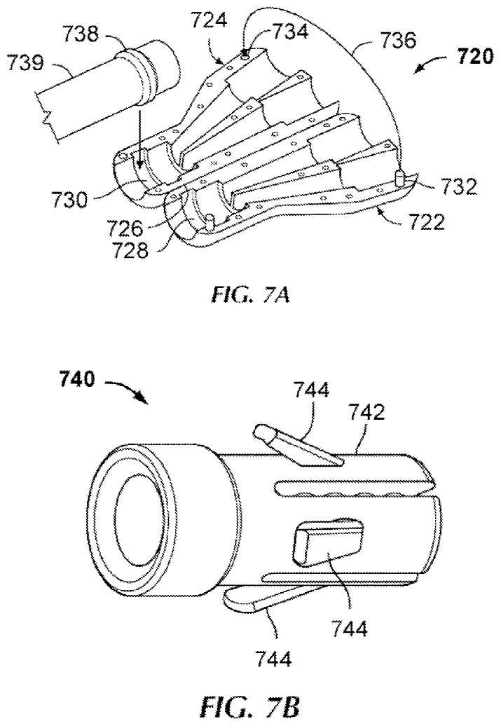

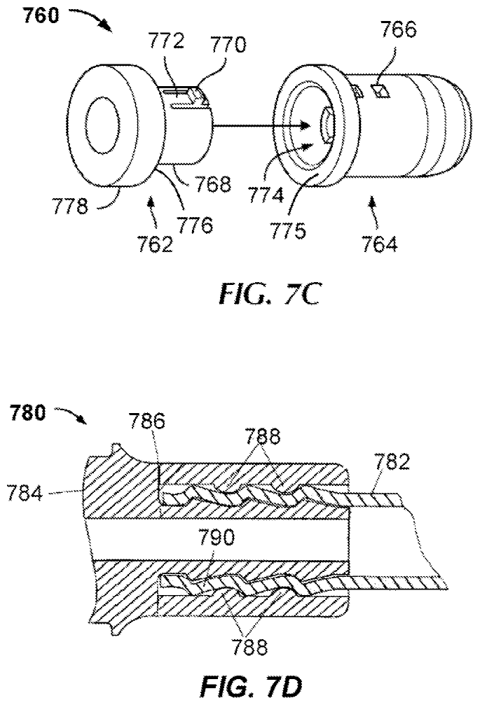

FIGS. 7A-D illustrate example removable catheter hubs, as constructed in accordance with at least one embodiment. FIG. 7A illustrates an example removable hub having a snap connection between two portions of the removable hub, as constructed in accordance with at least one embodiment. An example removable hub 720 is composed of two half shells 722 and 724 joined together by a hinge 726. Recesses 728 and 730 within the half shells 722 and 724, respectively, capture a protrusion at a proximal end 738 of a catheter body 739. To secure the catheter body 739 within the removable hub 720, the proximal end 738 of the catheter body 739 is positioned within one of the recesses 728 and 730, and one or both of the half shells 722 and 724 are moved transversely, as depicted by an arc 736, such that the two half shells 722 and 724 are joined together with a snap connection. As depicted, the half shell 722 includes a pin 732 and the half shell 724 includes a complimentary hole 734 to capture the pin 732 when the removable hub 720 is secured to the catheter body 739. The removable hub 720 can be removed by transversely moving the half shells 722 and 724 relative to each other, such that the pin 732 disengages the complimentary hole 734, and removing the catheter body 739 from the removable hub 720.

In some implementations, two portions of a removable hub are joined together by a snap-fit connection that can be disengaged with an axial movement between a first and second portion of the removable hub. FIG. 7B illustrates a first portion of an example removable hub having a deflectable projection, as constructed in accordance with at least one embodiment. An example first portion 740 of a removable hub includes a tubular member 742 and deflectable projections 744. A mating portion (not pictured) can receive the tubular member 742 of the first portion 740 and apply an external force on the deflectable projections 744 to lock the tubular member 742 within the mating portion. To remove the removable hub, the first portion 740 of the removable hub is removed from the mating portion by axially moving the first portion 740 in a direction proximal to the mating portion.

In some implementations, a deflectable projection of a portion of a removable hub includes a live hinge with a locking tab. FIG. 7C illustrates an example removable hub having a collet lock, as constructed in accordance with at least one embodiment. An example removable hub 760 includes a lockable insert 762 and a collet sleeve 764. The lockable insert 762 can include a flange 778 and a stem 768. The stem 768 includes a locking tab 770 at the end of a live hinge 772. The collet sleeve 764 includes a collet (not shown) deposited within a center channel 774 configured to receive the stem 768 of the lockable insert 762. When the stem 768 of the lockable insert 762 is received by the collet, at least a portion of the locking tab 770 engages a recess 766 of the collet sleeve 764. A surface 776 of the flange 778 may also abut a surface 775 of the collet sleeve 764 when the lockable insert 762 is inserted within the collet sleeve 764 such that the surfaces 775 and 776 act as stop surfaces to prevent axial movement of the collet sleeve 764 relative to the lockable insert 762. The lockable insert 762 can be removed from the collet sleeve 764 by compressing the locking tab 770 through the recess 766 of the collet sleeve 764 and moving the lockable insert 762 proximally relative to the collet sleeve 764.

In some implementations, a removable hub can be attached to a proximal end of a catheter body with a friction fit. FIG. 7D illustrates a cross sectional view of an example treatment catheter having a removable hub, as constructed in accordance with at least one embodiment. A treatment catheter 780 includes a catheter body 782 and a removable hub 784. The removable hub 784 includes a circumferential recess 786 to receive a proximal end 790 of the catheter body 782. The catheter body 782 can be flexible and can deform when inserted into the recess 786 of the removable hub 784. The recess 786 can include protrusions 788 extending radially towards the proximal end 790 of the catheter body 782 such that when the proximal end 790 is inserted into the recess 786, the catheter body 782 deforms and a frictional force between the protrusions 788 and the proximal end 790 securely attaches the catheter body 782 to the removable hub 784. To remove the removable hub 784 from the catheter body 782, an axial force is applied to the removable hub 784 to move the removable hub 784 proximally relative to the catheter body 782. The removable hub 784 can be reattached to the catheter body 782 by inserting the proximal end 790 into the recess 786.

Any of the removable hubs depicted in FIGS. 7A-D can be used with treatment catheters. For example, with reference to FIG. 4, the removable hub 452 can be a removable hub having a lever, as depicted in FIGS. 5A-B. As another example, with reference to FIG. 4, the removable hub 452 can be attached to the catheter body 450 using a threaded connection, as depicted in FIGS. 6A-B. The removable hub 452 can be any of the removable hubs depicted in FIGS. 7A-D. Removable hubs for use with treatment catheters are not limited to the removable hubs depicted across FIGS. 5A-B, 6A-B, and 7A-D and can be any removable hub that enables the advancement of a guide extension catheter over the catheter body of the treatment catheter when the removable hub is removed.

Treatment catheters with removable hubs used in tandem with guide extension catheters allow an operator to use a guide extension catheter without removing the treatment catheter after it has been positioned within a patient. When a treatment catheter is completely removed from the guide catheter in order to deliver a guide extension catheter, the treatment catheter must be repositioned after the guide extension catheter is positioned. As it can be difficult to position a treatment catheter in coronary arteries having complex geometry, this can extend the duration of a procedure, potentially requiring additional fluoroscopy and anesthesia. When used with a treatment catheter having a removable hub, the guide extension catheter can be delivered without risking the loss of the positioning of the treatment catheter, in turn shortening the duration of the procedure and decreasing risks associated with repositioning the treatment catheter.

FIG. 8 illustrates a plan view of the guide extension catheter of FIG. 3 used with a guide catheter, a treatment catheter having a removable hub, and a hemostasis valve, as constructed in accordance with at least one embodiment. A catheter system 800 can include a guide catheter 802, a treatment catheter 850, a hemostasis valve 862, and the guide extension catheter 318. The hemostasis valve 862 can be coupled to the guide catheter 802. FIG. 8 depicts the hemostasis valve 862 having a stem 863 that enters the guide catheter 802. However, the hemostasis valve 862 may be coupled to the guide catheter 802 by other means known by those skilled in the art. For example, the hemostasis valve 862 can be coupled to the guide catheter 802 with a rotating Luer lock. The hemostasis valve 862 can include a wing 864 configured to receive fluid and a linear branch 866 having an inner lumen. The linear branch 866 can be substantially coaxial with a longitudinal axis 860 of the guide catheter 802. A diameter of the inner lumen of the hemostasis valve 862 can be larger than an outer diameter of the guide extension catheter 318 to allow for insertion of the guide extension catheter 318 in the guide catheter 802.

The treatment catheter 850 can include a removable hub 852 at a proximal end 851 of the treatment catheter 850. FIG. 8 depicts the removable hub 852 as secured to the treatment catheter 850. However, at times during a procedure, the removable hub 852 may be removed from the treatment catheter 850 to allow for insertion of the guide extension catheter 318. The treatment catheter 850 can include an interventional device 870 on a distal portion of the treatment catheter 850. While the interventional device 870 is depicted as a balloon, the interventional device 870 can be a stent or any other interventional device for use in an angioplasty procedure, for example. When inserted within the guide catheter 802, the proximal end 851 of the treatment catheter 850 extends beyond a proximal end 803 of the guide catheter 802 and the hemostasis valve 862 to allow for an operator to slide the guide extension catheter 318 over the proximal end 851 of the treatment catheter 850. Once the guide extension catheter 318 is advanced through the guide catheter 802 over the treatment catheter 850, a distal portion of the guide extension catheter 318 exits a distal end 816 of the guide catheter 802. Then, the treatment catheter 850 can continue to be advanced into an artery with support provided by the guide extension catheter 318. The interventional device 870 can be inserted into the artery and positioned across a lesion via the treatment catheter 850.

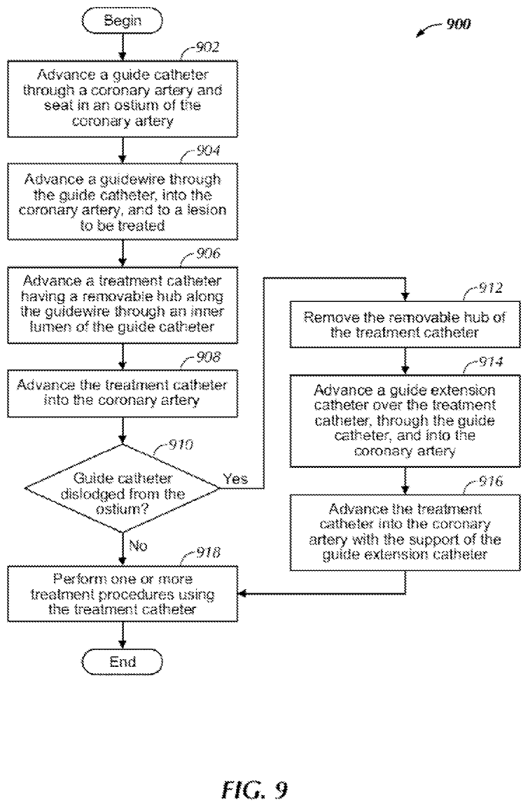

FIG. 9 is a flowchart illustrating a method for delivering a guide extension catheter over a prepositioned treatment catheter, in accordance with embodiments of the present invention. Operations of a flowchart depict a method 900 including the use of a guide extension catheter to treat a lesion in an artery after attempting to insert a treatment catheter into an artery or vessel where the lesion is located. The operations included in the method 900 may be performed by a person or persons, such as a physician and/or one or more qualified medical technicians.

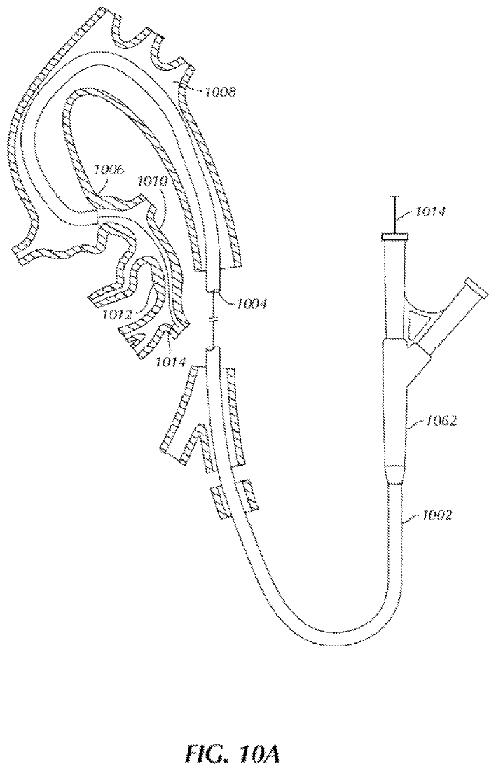

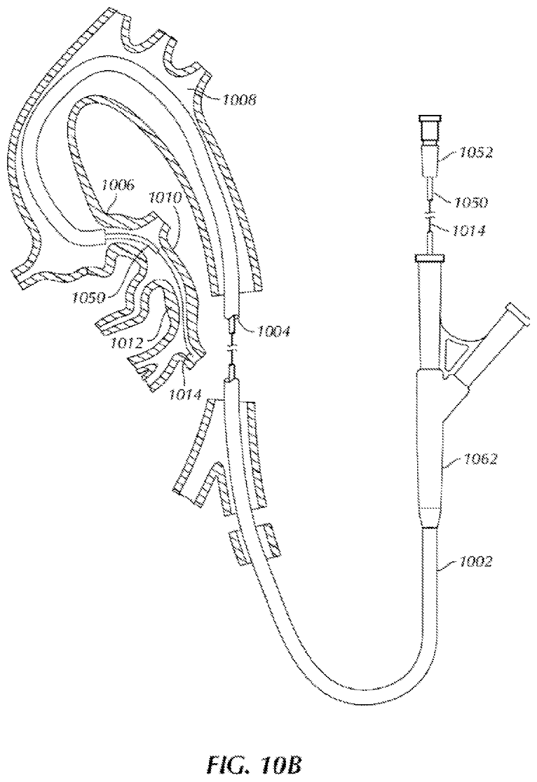

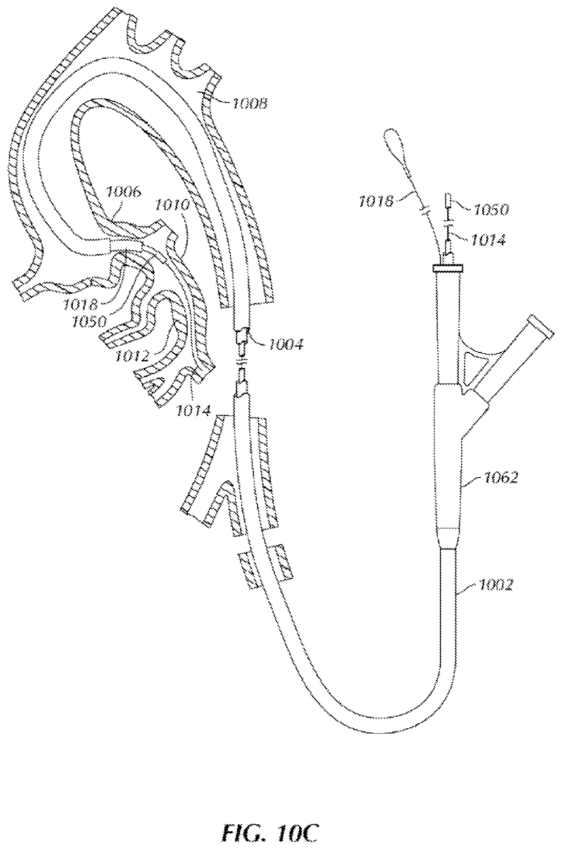

At block 902, embodiments of the method 900 include advancing a guide catheter through the aorta until a distal end of the guide catheter is seated in an ostium of a coronary artery. To help illustrate, FIG. 10A illustrates a guide catheter and guidewire positioned in a coronary artery. A guide catheter 1002 is advanced through an aorta 1008 until a distal end is seated in an ostium 1006 of a coronary artery 1010.

Referring again to FIG. 9, at block 904, embodiments of the method 900 include advancing a guidewire through an inner lumen of the guide catheter until a distal portion of the guidewire exits the distal end of the guide catheter and enters into the coronary artery and crosses a lesion to be treated. For example, with reference to FIG. 10A, a guidewire 1014 is advanced through a hemostasis valve 1062 and through a lumen 1004 of the guide catheter 1002 until a distal end of the guidewire 1014 exits the guide catheter 1002 into the coronary artery 1010.

Referring again to FIG. 9, at block 906, embodiments of the method 900 include inserting a treatment catheter having a removable hub at a proximal end over the guidewire 1014 and advancing the treatment catheter along the guidewire and through the lumen 1004 of the guide catheter such that the proximal end of the treatment catheter extends from a proximal end of the guide catheter. To help illustrate, FIG. 10B illustrates a treatment catheter having a removable hub positioned in a guide catheter in a coronary artery. A treatment catheter 1050 having a removable hub 1052 is advanced over a proximal end of the guidewire 1014 and into the guide catheter 1002 through the hemostasis valve 1062.

Referring again to FIG. 9, at block 908, embodiments of the method 90M include advancing a distal portion of the treatment catheter beyond the distal end of the guide catheter and into the coronary artery. In some embodiments, the treatment catheter can be a torqueable microcatheter and advancing the distal portion of the treatment catheter can include rotating the treatment catheter is a clockwise or counterclockwise direction. For example, with reference to FIG. 4, the screw portion 454 of the treatment catheter 400 can include a helical thread structure projecting from the outer surface of the catheter body 450 and can engage a blockage within an artery.

Referring again to FIG. 9, at block 910, embodiments of the method 900 include determining whether advancing the treatment catheter further into the coronary artery dislodges the distal end of the guide catheter from the ostium. If a determination is made that advancing the treatment catheter further into the coronary artery does not dislodge the distal end of the guide catheter from the ostium (“NO” arrow extending from block 910), embodiments of the method 900 proceed to block 918. If a determination is made at block 910 that advancing the treatment catheter further into the coronary artery does dislodge the distal end of the guide catheter from the ostium (“YES” arrow extending from block 910), embodiments of the method 900 proceed to block 912.

At block 912, embodiments of the method 900 include removing the removable hub of the treatment catheter. For example, with reference to FIG. 103, the removable hub 1052 of the treatment catheter 1050 can be removed. The removable hub is not limited to any particular type of removable hub, and in various embodiments includes a hub as illustrated and described above with respect to any of FIGS. 5A-B, 6A-B, and 7A-D.

Referring again to FIG. 9, at block 914, embodiments of the method 900 include advancing a guide extension catheter over the hubless proximal end of the treatment catheter and through the lumen of the guide catheter until a distal end of the guide extension catheter exits the distal end of the guide catheter and extends into the coronary artery. To help illustrate. FIG. 10C depicts a guidewire, a treatment catheter, and a guide extension catheter arrange in a guide catheter. A guide extension catheter 1018 is advanced over the treatment catheter 1050 and through the lumen 1004 of the guide catheter 1002 such that at least a portion of the treatment catheter 1050 is interior to a tubular distal portion of the guide extension catheter 1018 and a portion of the guide extension catheter 1018 is internal to the guide catheter 1002. The tubular distal portion of the guide extension catheter 1018 is further advanced through the lumen of the guide catheter 1002 until a distal portion of the guide extension catheter exits the distal end of the guide catheter 1002 and extends into the coronary artery 1010. Optionally, a distal end of the tubular distal portion of the guide extension catheter 1018 can include a side opening (e.g., have an angled opening) to facilitate a balloon, stent or other treatment device located on the treatment catheter being retracted into or received by the tubular distal portion during use of the guide extension catheter 1018.

Referring again to FIG. 9, at block 916, embodiments of method 900 include advancing the treatment catheter into the coronary artery with the support of the guide extension catheter.

Referring again to FIG. 9, at block 918, embodiments of the method 900 include performing one or more treatment procedures using the treatment catheter. For example, performing a treatment procedure can include advancing an interventional device, positioned on a distal portion of the treatment catheter, within the lesion. For example, a balloon or stent can be positioned at the distal end of the treatment catheter 1050 and can be advanced to a lesion 1012 via the treatment catheter 1050. Following completion of the treatment procedures, the method 900 may be completed.

The flowchart depicting the method 900 is provided to aid in understanding the illustrations and is not to be used to limit scope of the claims. The flowchart depicts example operations that can vary within the scope of the claims. Additional operations may be performed; fewer operations may be performed; the operations may be performed in parallel; and the operations may be performed in a different order with the exception of the removable hub being removed from the treatment catheter prior to initial advancement of the guide extension catheter over the treatment catheter.

While the aspects of the disclosure are described with reference to various implementations and exploitations, it will be understood that these aspects are illustrative and that the scope of the claims is not limited to them. In general, techniques for delivering guide extension catheters via a removable hub of a treatment catheter as described herein may be implemented with facilities consistent with any interventional device or treatment catheter having a removable hub that, when removed, permits a catheter having a larger inner diameter than an outer diameter of the treatment catheter to pass over the treatment catheter. Many variations, modifications, additions, and improvements are possible.

### Closing Notes:

The above Detailed Description includes references to the accompanying drawings, which form a part of the Detailed Description. The Detailed Description should be read with reference to the drawings. The drawings show, by way of illustration, specific embodiments in which the present systems, devices, and methods can be practiced. These embodiments are also referred to herein as “examples.”

The above Detailed Description is intended to be illustrative and not restrictive. For example, the above-described examples (or one or more features or components thereof) can be used in combination with each other. Other embodiments can be used, such as by one of ordinary skill in the art upon reviewing the above Detailed Description. Also, various features or components have been or can be grouped together to streamline the disclosure. This should not be interpreted as intending that an unclaimed disclosed feature is essential to any claim. Rather, inventive subject matter can lie in less than all features of a particular disclosed embodiment. Thus, the following claim examples are hereby incorporated into the Detailed Description, with each example standing on its own as a separate embodiment:
In Example 1, a method for using a guide extension catheter with a prepositioned treatment catheter comprises positioning a treatment catheter over a guidewire and through a guide catheter such that a proximal end of the treatment catheter extends from a proximal end of the guide catheter, removing a removable hub from the proximal hub of the treatment catheter, advancing a guide extension catheter having a tubular distal portion through the guide catheter and along an outer surface of the treatment catheter, including sliding the tubular distal portion of the guide extension catheter over the hubless proximal end of the treatment catheter, and reattaching the removable hub to the proximal end of the treatment catheter.
In Example 2, the method comprises the method of Example 1, wherein removing the removable hub from the proximal end of the treatment catheter includes rotating the removable hub relative to the treatment catheter.
In Example 3, the method comprises the method of Example 1, wherein removing the removable hub from the proximal end of the treatment catheter includes moving a lever in a direction parallel to an axis defined by the proximal end of the treatment catheter.
In Example 4, the method comprises the method of Example 1, wherein removing the removable hub from the proximal end of the treatment catheter includes disengaging a snap-fit connection between portions of the removable hub.
In Example 5, the method comprises the method of Example 4, wherein disengaging the snap-fit connection between portions of the removable hub includes an axial movement between first and second portions of the removable hub.
In Example 6, the method comprises the method of Example 5, wherein the first portion of the removable hub includes a deflectable projection, and wherein the second portion of the removable hub includes an opening to receive the deflectable projection.
In Example 7, the method comprises the method of Example 4, wherein disengaging the snap-fit connection between portions of the removable hub includes a transverse movement between first and second portions of the removable hub.
In Example 8, the method comprises the method of Example 1, wherein removing the removable hub from the proximal end of the treatment catheter includes overcoming a friction fit between a portion of the removable hub and a portion of the treatment catheter.
In Example 9, the method comprises the method of any one of Examples 1-8, wherein reattaching the removable hub to the proximal end of the treatment catheter includes securing the removable hub to the treatment catheter such that relative axial movement is inhibited.
In Example 10, the method comprises the method of any one of Examples 1-8, wherein reattaching the removable hub to the proximal end of the treatment catheter includes securing the removable hub to the treatment catheter such that relative transverse movement is inhibited.
In Example 11, the method comprises the method of any one of Examples 1-8, wherein reattaching the removable hub to the proximal end of the treatment catheter includes establishing a fluid tight connection between the removable hub and the treatment catheter.
In Example 12, the method comprises the method of any one of Examples 1-11, further comprising positioning a distal end of the guide catheter at an ostium of a coronary artery.
In Example 13, the method comprises the method of Example 12, further comprising advancing the treatment catheter over the guidewire and through the guide catheter to a treatment site within the coronary artery, and wherein such advancement causes the distal end of the guide catheter to back away from the ostium of the coronary artery.
In Example 14, the method comprises the method of any one of Examples 1-13, wherein advancing the guide extension catheter through the guide catheter and along the outer surface of the treatment catheter further includes advancing a portion of the tubular distal portion of the guide extension catheter beyond the distal end of the guide catheter and into a coronary artery.
In Example 15, the method comprises the method of Example 14, further comprising advancing the treatment catheter to a treatment site in the coronary artery.
In Example 16, the method comprises the method of Example 15, wherein advancing the treatment catheter to the treatment site includes delivering one or both of an inflatable balloon and a stent, positioned along a distal portion of the treatment catheter, to the treatment site with the support of the guide extension catheter.
In Example 17, the method comprises the method of Example 15, further comprising advancing the guidewire through a blockage at the treatment site with the support of the treatment catheter and the guide extension catheter.
In Example 18, the method comprises the method of Example 17, further comprising advancing the treatment catheter through the blockage, including rotating the treatment catheter in a clockwise or counterclockwise direction.
In Example 19, the method comprises the method of Example 18, wherein a distal portion of the treatment catheter comprises a helical threat structure projecting from its outer surface and configured to engage the blockage.
In Example 20, the method comprises the method of any one of Examples 1-19, wherein the guide extension catheter is a rapid exchange catheter.
In Example 21, the method comprises the method of any one of Examples 1-19, wherein the guide extension catheter is an over-the-wire catheter.
In Example 22, a treatment catheter comprises a catheter body and a removable hub secured to a proximal end of the catheter body, wherein the removable hub is removable from the catheter body to allow for advancement of a guide extension catheter over the proximal end of the catheter body.
In Example 23, the treatment catheter comprises the treatment catheter of Example 22, wherein the catheter body is tubular, and wherein an outer diameter of the catheter body is less than an inner diameter of a tubular guide catheter in which the treatment catheter is to be inserted.
In Example 24, the treatment catheter comprises the treatment catheter of Example 22 or 23, wherein the catheter body is tubular, and wherein an outer diameter of the catheter body is less than an inner diameter of a tubular distal portion of the guide extension catheter.
In Example 25, the treatment catheter comprises the treatment catheter of any one of Examples 22-24, wherein the removable hub is secured to the catheter body using a threaded connection.
In Example 26, the treatment catheter comprises the treatment catheter of Example 25, wherein the removable hub comprises an internally threaded portion, and wherein the proximal end of the catheter body comprises an externally threaded portion.
In Example 27, the treatment catheter comprises the treatment catheter of any one of Examples 22-24, wherein the removable hub comprises a collet lock.
In Example 28, the treatment catheter comprises the treatment catheter of any one of Examples 22-27, wherein the removable hub comprises a stem, and wherein the catheter body is configured to receive the stem of the removable hub.
In Example 29, the treatment catheter comprises the treatment catheter of any one of Examples 22-28, wherein the distal portion of the catheter body includes a balloon, and wherein the catheter body comprises a seal positioned at the proximal end of the catheter body and internal to the catheter body to maintain an inflation fluid pressure within the balloon when the removable hub is removed.
In Example 30, the treatment catheter comprises the treatment catheter of any one of Examples 22-29, wherein a distal portion of the catheter body comprises a helical threat structure projecting from its outer surface.

1-35. (canceled)

36. A method, comprising:
positioning a treatment catheter over a guidewire and through a guide catheter such that a proximal end of the treatment catheter extends from a proximal end of the guide catheter;
removing a removable hub from the proximal end of the treatment catheter; advancing a guide extension catheter having a tubular distal portion through the guide catheter and along an outer surface of the treatment catheter, including sliding the tubular distal portion of the guide extension catheter over a hubless proximal end of the treatment catheter, and
reattaching the removable hub to the proximal end of the treatment catheter,
wherein the inserted treatment catheter is not removed prior to the advancing of the guide extension catheter through the guide catheter.

37. The method of claim 36, wherein removing the removable hub from the proximal end of the treatment catheter comprises:
rotating the removable hub relative to the treatment catheter; or
moving a lever in a direction parallel to an axis defined by the proximal end of the treatment catheter; or
disengaging a snap-fit connection between two or more portions of the removable hub; or
overcoming a friction fit between a portion of the removable hub and a portion of the treatment catheter.

38. The method of claim 37, wherein disengaging the snap-fit connection between the two or more portions of the removable hub includes an axial movement between a first portion of the two or more portions and a second portion of the two or more portions of the removable hub.

39. The method of claim 38, wherein the first portion of the removable hub includes a deflectable projection, and wherein the second portion of the removable hub includes an opening to receive the deflectable projection.

40. The method of claim 37, wherein disengaging the snap-fit connection between portions of the removable hub includes a transverse movement between first and second portions of the removable hub.

41. The method of claim 36, wherein reattaching the removable hub to the proximal end of the treatment catheter comprises:
securing the removable hub to the treatment catheter such that relative axial movement in inhibited; or
securing the removable hub to the treatment catheter such that relative transverse movement is inhibited; or
establishing a fluid tight connection between the removable hub and the treatment catheter.

42. The method of claim 36, further comprising positioning a distal end of the guide catheter at an ostium of a coronary artery.

43. The method of claim 42, further comprising advancing the treatment catheter over the guidewire and through the guide catheter to a treatment site within the coronary artery, and wherein such advancement causes the distal end of the guide catheter to back away from the ostium of the coronary artery.

44. The method of claim 36, wherein advancing the guide extension catheter through the guide catheter and along the outer surface of the treatment catheter further comprises advancing a portion of the tubular distal portion of the guide extension catheter beyond a distal end of the guide catheter and into a coronary artery; optionally further comprising advancing the treatment catheter to a treatment site in the coronary artery.

45. The method of claim 44, wherein advancing the treatment catheter to the treatment site includes delivering one or both of an inflatable balloon or a stent, positioned along a distal portion of the treatment catheter, to the treatment site with support of the guide extension catheter.

46. The method of claim 44, further comprising advancing the guidewire through a blockage at the treatment site with support of the treatment catheter and the guide extension catheter.

47. The method of claim 46, further comprising advancing the treatment catheter through the blockage, including rotating the treatment catheter in a clockwise or counterclockwise direction.

48. The method of claim 47, wherein a distal portion of the treatment catheter comprises a helical thread structure projecting from an outer surface and configured to engage the blockage.

49. The method of claim 36, wherein the guide extension catheter is a rapid exchange catheter or an over-the-wire catheter.

50. The method of claim 36, for use in treating:
a vessel, wherein the vessel has a lesion, a blockage, or is diseased; and/or
a hollow structure, wherein the hollow structure is a biliary tract or ureter.

51. A treatment catheter, comprising:
a catheter body; and
a removable hub secured to a proximal end of the catheter body, wherein the removable hub is removable from the catheter body to allow for advancement of a guide extension catheter over the proximal end of the catheter body.

52. The treatment catheter of claim 51, wherein:
the catheter body is tubular, and wherein an outer diameter of the catheter body is less than an inner diameter of a tubular guide catheter in which the treatment catheter is to be inserted; and/or
the catheter body is tubular, and wherein an outer diameter of the catheter body is less than an inner diameter of a tubular distal portion of the guide extension catheter.

53. The treatment catheter of claim 51, wherein the removable hub is secured to the catheter body using a threaded connection, wherein the removable hub comprises an internally threaded portion, and wherein the proximal end of the catheter body comprises an externally threaded portion.

54. The treatment catheter of claim 51, wherein:
the removable hub comprises a collet lock; and/or
wherein the removable hub comprises a stem, and wherein the catheter body is configured to receive the stem of the removable hub.

55. The treatment catheter of claim 51, wherein:
a distal portion of the catheter body includes a balloon, and wherein the catheter body comprises a seal positioned at the proximal end of the catheter body and internal to the catheter body to maintain an inflation fluid pressure within the balloon when the removable hub is removed; and/or
wherein a distal portion of the catheter body comprises a helical thread structure projecting from its outer surface.

56. The treatment catheter of claim 51, comprising the guide extension catheter over the proximal end of the catheter body.

57. A catheter system comprising a treatment catheter, guide catheter and guide extension catheter:
the treatment catheter comprising a catheter body and a removable hub secured to a proximal end of the catheter body, wherein the removable hub is removable from the catheter body to allow for advancement of a guide extension catheter over the proximal end of the catheter body,
the treatment catheter is for positioning over a guidewire and through a guide catheter such that a proximal end of the treatment catheter extends from a proximal end of the guide catheter,
wherein a removable hub is removable from the proximal end of the treatment catheter,
wherein the guide extension catheter having a tubular distal portion is for advancing through the guide catheter and along an outer surface of the treatment catheter, without removal of the treatment catheter, including sliding the tubular distal portion of the guide extension catheter over a hubless proximal end of the treatment catheter, and
wherein the removable hub is re-attachable to the proximal end of the treatment catheter.
